(19) United States
(12) Patent Application Publication
(10) Pub. No.: US 2004/0126765 A1
(43) Pub. Date: Jul. 1, 2004

(54) Method and compositions for sequencing nucleic acid molecules

(76) Inventor: Craig W. Adams, Yorba Linda, CA (US)

Correspondence Address: LINIAK, BERENATO & WHITE, LLC, 6550 ROCK SPRING DRIVE, SUITE 240, BETHESDA, MD, 20817, US

(21) Appl. No.: 10/329,752

(22) Filed: Dec. 27, 2002

(51) Int. Cl. C12Q 1/68; C12P 19/34

(52) U.S. Cl. 435/6; 435/91.2

(57) ABSTRACT

The invention relates to methods, compositions, kits and apparati for sequencing nucleic acid molecules. The invention particularly concerns the use of an exonuclease activity in concert with a polymerase activity to mediate such sequencing.

### FIELD OF THE INVENTION

The invention relates to methods, compositions, kits and apparati for sequencing nucleic acid molecules. The invention particularly concerns the use of an exonuclease activity in concert with a polymerase activity to mediate such sequencing.

### BACKGROUND OF THE INVENTION

The capability of determining the sequences of nucleic acid molecules is of fundamental importance to modern biology and medicine (Glasel J A. (2002) “DRUGS, THE HUMAN GENOME, AND INDIVIDUAL-BASED MEDICINE,” Prog. Drug Res. 58:1-50; Green, E. D. (2001) “Strategies for the Systemic Sequencing of Complex Genomes,” Nat. Rev. Genet. 2:6-12; Opalinska, J. B. et al. (2002) “NUCLEIC-ACID THERAPEUTICS: BASIC PRINCIPLES AND RECENT APPLICATIONS,” Nat. Rev. Drug Discov. 1:503-514; Kim, Y. et al. (2002) “THE NUCLEOTIDE: DNA SEQUENCING AND ITS CLINICAL APPLICATION,” J. Oral Maxillofac. Surg. 60:924-930).

Initial attempts to determine the sequence of a DNA molecule involved extensions of techniques that had been initially developed to permit the sequencing of RNA molecules (Sanger, F. (1965) “A TWO-DIMENSIONAL FRACTIONATION PROCEDURE FOR RADIOACTIVE NUCLEOTIDES,” J. Mol. Biol. 13:373-398; Brownlee, G. G. et al. (1968) “THE SEQUENCE OF 5 S RIBOSOMAL RIBONUCLEIC ACID,” J. Molec. Biol. 34:379-412). Such methods exploited the specific cleavage of DNA into smaller fragments by (1) enzymatic digestion (Robertson, H. D. et al. (1973) “ISOLATION AND SEQUENCE ANALYSIS OF A RIBOSOME-PROTECTED FRAGMENT FROM BACTERIOPHAGE ΦX174 DNA,” Nature New Biol. 241:38-40; Ziff, E. B. et al. (1973) “DETERMINATION OF THE NUCLEOTIDE SEQUENCE OF A FRAGMENT OF BACTERIOPHAGE ΦX174 DNA,” Nature New Biol. 241:34-37); (2) nearest neighbor analysis (Wu, R. et al. (1971) “Nucleotide Sequence Analysis Of DNA. 1′. Complete Nucleotide Sequence Of The Cohesive Ends Of Bacteriophage Lambda DNA,” J. Molec. Biol. 57:491-511), or (3) the “Wandering SPOT” method (Sanger, F. (1973) “USE OF DNA POLYMERASE I PRIMED BY A SYNTHETIC OLIGONUCLEOTIDE TO DETERMINE A NUCLEOTIDE SEQUENCE IN PHAGE FL DNA,” Proc. Natl. Acad. Sci. (U.S.A.) 70:1209-1213 (1973).

The most commonly used methods of nucleic acid sequencing comprise the “dideoxy-mediated chain termination method,” also known as the “Sanger Method” (Sanger, F. et al. (1975) “A RAPID METHOD FOR DETERMINING SEQUENCES IN DNA BY PRIMED SYNTHESIS WITH DNA POLYMERASE,” J. Molec. Biol. 94:441-448 (1975); Sanger, F. et al. (1977) “DNA SEQUENCING WITH CHAIN-TERMINATING INHIBITORS,” Proc. Natl. Acad. Sci. (USA) 74:5463-5467; Prober, J. et al. “A SYSTEM FOR RAPID DNA SEQUENCING WITH FLUORESCENT CHAIN-TERMINATING DIDEOXYNUCLEOTIDES,” (1987) Science 238:336-341 (1987)) and the “chemical degradation method,” also known as the “Maxam-Gilbert method” (Maxam, A. M. et al. (1977) “NEW METHOD FOR SEQUENCING DNA.,” Proc. Natl. Acad. Sci. (U.S.A.) 74:560-564). Methods for sequencing DNA using either the dideoxy-mediated method or the Maxam-Gilbert method are widely known to those of ordinary skill in the art. Such methods are, for example, disclosed in Maniatis, T. et al. (1989) “MOLECULAR CLONING, A LABORATORY MANUAL, 2nd Edition,” Cold Spring Harbor Press, Cold Spring Harbor, N.Y., and in Zyskind, J. W. et al. (1988) RECOMBINANT DNA LABORATORY MANUAL, Academic Press, Inc., New York. Methods of DNA sequencing are reviewed by Marziali, A. et al. (2001) (“NEW DNA SEQUENCING METHODS,” Ann. Rev. Biomed. Eng. 3:195-223), Graham, C. A. et al. (2001) (“INTRODUCTION TO DNA SEQUENCING,” Methods Molec. Biol. 167:1-12), Messing, J. (2001) (“THE UNIVERSAL PRIMERS AND THE SHOTGUN DNA SEQUENCING METHOD,” Methods Molec. Biol. 167:13-31) and Bankier, A. T. (2001) (“SHOTGUN DNA SEQUENCING,” Methods Molec. Biol. 167:89-100).

The Maxam-Gilbert method of DNA sequencing is a degradative method in which a fragment of DNA is labeled at one end (or terminus) and partially cleaved in four separate chemical reactions, each of which is specific for cleaving the DNA molecule at a particular base (G or C) at a particular type of base (A/G, C/T, or A>C). The effect of such reactions is to create a set of nested molecules whose lengths are determined by the locations of a particular base along the length of the DNA molecule being sequenced. The nested reaction products are then resolved by electrophoresis, and the end-labeled molecules are detected, typically by autoradiography when a ³²P label is employed. Four single lanes are typically required in order to determine the sequence. Although the Maxam-Gilbert method uses simple chemical reagents which are readily available, it is extremely laborious to perform and requires meticulous experimental technique.

Owing to these deficiencies, the dideoxy-mediated or “Sanger” chain termination method of DNA sequencing has become the method of choice. In the dideoxy-mediated sequencing method, the sequence of a DNA molecule is obtained through the extension of an oligonucleotide primer that is hybridized to the nucleic acid molecule being sequenced. In brief, four separate primer extension reactions are conducted. In each reaction, a DNA polymerase is added along with the four nucleotide triphosphates (dATP, dCTP, dGTP, and dTTP) needed to polymerize DNA. Significantly, each reaction also contains a 2′,3′ dideoxy derivative of the dATP, dCTP, dGTP, or dTTP nucleotides. Such derivatives differ from conventional nucleotides in lacking a hydroxyl residue at the 3′ position of deoxyribose. Although DNA polymerases can incorporate a dideoxy nucleotide into the primer extension product, such incorporation blocks further primer extension. Thus, the incorporation of a dideoxy derivative results in the termination of the extension reaction.

By conducting the dideoxy sequencing reaction under conditions in which the dideoxy nucleotides are present in lower concentrations than their corresponding conventional nucleotides, the net result of each of the four reactions is the production of a nested set of oligonucleotides, each of which is terminated by the particular dideoxy derivative used in the reaction. By subjecting the reaction products of each of the extension reactions to electrophoresis, it is possible to obtain a series of four “ladders,” of bands. Since the position of each “rung” of the ladder is determined by the size of the molecule, and since such size is determined by the incorporation of the dideoxy derivative, the appearance and location of a particular “rung” can be readily translated into the sequence of the extended primer. Thus, the sequence of the extended primer can be determined through electrophoretic analysis.

The adoption of the Sanger method as the method of choice was spurred by the development of novel polymerases that could more readily incorporate fluorescent and other non-radioactively labeled dideoxynucleotides (Tabor, S. et al. (1995) “A SINGLE RESIDUE IN DNA POLYMERASES OF THE ESCHERICHIA COLI DNA POLYMERASE I FAMILY IS CRITICAL FOR DISTINGUISHING BETWEEN DEOXY- AND DIDEOXYRIBONUCLEOTIDES,” Proc. Natl. Acad. Sci. USA 92, 6339-6343; Tabor, S. et al. (U.S. Pat. No. 5,614,365, U.S. Pat. No. 5,674,716).

As originally implemented, the “Sanger” method required separate sequencing reactions for each of the four possible nucleotides. One alternative to this requirement was developed by Prober, J. M. et al., who developed differentially labeled dideoxynucleoside triphosphates. The use of such reagents enables the sequencing reaction to be conducted in a single reaction tube (Prober, J. M. et al. (1987) “A SYSTEM FOR RAPID DNA SEQUENCING WITH FLUORESCENT CHAIN-TERMINATING DIDEOXYNUCLEOTIDES,” Science 238:336-341; Prober, et al. (U.S. Pat. No. 5,242,796); Prober, et al. (U.S. Pat. No. 5,306,618); Prober, et al. (U.S. Pat. No. 5,332,666); Lee, L. G. et al. (1992) discloses the use of dye-labeled terminators, and their incorporation into DNA by T7 polymerase (Lee, L. G. et al. (1992) “DNA SEQUENCING WITH DYE-LABELED TERMINATORS AND T7 DNA POLYMERASE: EFFECT OF DYES AND DNTPs ON INCORPORATION OF DYE-TERMINATORS AND PROBABILITY ANALYSIS OF TERMINATION FRAGMENTS,” Nucl. Acids Res. 20:2471-2483).

An essential characteristic of the “Sanger” method is the inclusion of conventional nucleotides and chain-terminator nucleotides in the same sequencing reaction. The inclusion of such a combination of nucleotide species is necessary in order to form the nested set of primer extension molecules that is required by the method. A variety of “microsequencing” methods have, however, been developed that employ fewer than all four conventional nucleotides, or that employ subsets of conventional and/or chain terminator nucleotide species. Such methods are employed in sequencing single nucleotide polymorphisms, and in conjunction with the use of random or pseudo-random ordered arrays of oligonucleotides.

For example, some such methods rely on the incorporation of labeled deoxynucleotides to discriminate between bases at a polymorphic site (Kornher, J. S. et al. (1989) “MUTATION DETECTION USING NUCLEOTIDE ANALOGS THAT ALTER ELECTROPHORETIC MOBILITY,” Nucl. Acids Res. 17:7779-7784; Sokolov, B. P. (1990) “PRIMER EXTENSION TECHNIQUE FOR THE DETECTION OF SINGLE NUCLEOTIDE IN GENOMIC DNA,” Nucl. Acids Res. 18:3671; Syvanen, A.-C., et al. (1990) “A PRIMER-GUIDED NUCLEOTIDE INCORPORATION ASSAY IN THE GENOTYPING OF APOLIPOPROTEIN E,” Genomics 8:684-692; Bajaj et al. (U.S. Pat. No. 5,846,710); Kuppuswamy, M. N. et al. (1991) “SINGLE NUCLEOTIDE PRIMER EXTENSION TO DETECT GENETIC DISEASES: EXPERIMENTAL APPLICATION TO HEMOPHILLIA B (FACTOR IX) AND CYSTIC FIBROSIS GENES,” Proc. Natl. Acad. Sci. (U.S.A.) 88:1143-1147; Prezant, T. R. et al. (1992) “TRAPPED-OLIGONUCLEOTIDE NUCLEOTIDE INCORPORATION (TONI) ASSAY, A SIMPLE METHOD FOR SCREENING POINT MUTATIONS,” Hum. Mutat. 1: 159-164; Ugozzoli, L. et al. (1992) “DETECTION OF SPECIFIC ALLELES BY USING ALLELE-SPECIFIC PRIMER EXTENSION FOLLOWED BY CAPTURE ON SOLID SUPPORT,” GATA 9:107-112; Nyren, P. et al. (1993) “SOLID PHASE DNA MINISEQUENCING BY AN ENZYMATIC LUMINOMETRIC INORGANIC PYROPHOSPHATE DETECTION ASSAY,” Anal. Biochem. 208:171-175; and Wallace (WO89/10414). Alternate methods involve combinations of conventional and chain-terminating nucleotides (Syvanen, A.-C. et al. (1993) “IDENTIFICATION OF INDIVIDUALS BY ANALYSIS OF BIALLELIC DNA MARKERS, USING PCR AND SOLID-PHASE MINISEQUENCING,” Amer. J. Hum. Genet. 52:46-59 (1993); Soderlund et al. (U.S. Pat. No. 6,013,431); Kornher, J. S. et al. (1989) “MUTATION DETECTION USING NUCLEOTIDE ANALOGS THAT ALTER ELECTROPHORETIC MOBILITY,” Nucl. Acids Res. 17:7779-7784). Other methods require the presence of chain-terminator nucleotides and the absence of conventional nucleotides (Goelet, P. et al. (WO 92/15712, U.S. Pat. No. 6,004,744, U.S. Pat. No. 5,952,174, U.S. Pat. No. 5,888,819).

Goelet, P. et al. (U.S. Pat. No. 5,888,819), for example, concerns a method for determining the identity of a nucleotide base at a specific position in a nucleic acid of interest in which a sample containing the nucleic acid of interest, in single-stranded form, is contacted with an oligonucleotide primer that is fully complementary to and which hybridizes specifically to a stretch of nucleotide bases of the nucleic acid of interest immediately adjacent to the nucleotide base to be identified, under high stringency hybridization conditions, so as to form a double-stranded nucleic acid molecule in which the nucleotide base to be identified is the first unpaired base in the template immediately downstream of the 3′ end of the primer. The double-stranded molecule is incubated, in the absence of non-chain terminator nucleotides, with at least two different chain terminator nucleotides, and in the presence of a polymerase, under conditions sufficient to cause a template-dependent, primer extension reaction to occur that is strictly dependent upon the identity of the unpaired nucleotide base in the template immediately downstream of the 3′ end of the primer. The identity of the nucleotide base to be identified is determined by detecting the identity of the incorporated chain-terminator nucleotide.

Mundy, C. R. (U.S. Pat. No. 4,656,127) discusses an alternative microsequencing method that employs a specialized exonuclease resistant nucleotide derivative. A primer complementary to an allelic sequence immediately 3′-to the polymorphic site is permitted to hybridize to a target molecule obtained from a particular animal or human. If the polymorphic site on the target molecule contains a nucleotide that is complementary to the particular exonucleotide-resistant nucleotide derivative present, then that derivative will be incorporated by a polymerase onto the end of the hybridized primer. Such incorporation renders the primer resistant to exonuclease, and thereby permits its detection. Since the identity of the exonucleotide-resistant derivative of the sample is known, a finding that the primer has become resistant to exonucleases reveals that the nucleotide present in the polymorphic site of the target molecule was complementary to that of the nucleotide derivative used in the reaction. Mundy's method has the advantage that it does not require the determination of large amounts of extraneous sequence data. It has the disadvantages of destroying the amplified target sequences, and unmodified primer and of being extremely sensitive to the rate of polymerase incorporation of the specific exonuclease resistant nucleotide being used.

Cohen, D. et al. (French Patent 2,650,840; WO91/02087) discuss a solution-based method for determining the identity of the nucleotide of a polymorphic site. As in the method of Mundy (U.S. Pat. No. 4,656,127), a primer is employed that is complementary to allelic sequences immediately 3′-to a polymorphic site. The method determines the identity of the nucleotide of that site using labeled dideoxynucleotide derivatives, which, if complementary to the nucleotide of the polymorphic site will become incorporated onto the terminus of the primer. Cheesman, P. (U.S. Pat. No. 5,302,509) describes a method for sequencing a single stranded DNA molecule using fluorescently labeled 3′-blocked nucleotide triphosphates. An apparatus for the separation, concentration and detection of a DNA molecule in a liquid sample has been recently described by Ritterband, et al. (PCT Patent Application No. WO95/17676). Dower, W. J. et al. (U.S. Pat. No. 5,547,839) describes a method for sequencing an immobilized primer using fluorescent labels. Chee, M. et al. (WO95/11995) describes an array of primers immobilized onto a solid surface. Chee et al. further describes a method for determining the presence of a mutation in a target sequence by comparing against a reference sequence with a known sequence.

In a further variation of such methods, ordered arrays of solid-phase bound random or pseudorandom oligonucleotides to function as primers for the sequencing reaction. In brief, such methods avoid the need for obtaining nested sets of fragments by hybridizing the intact molecule being sequenced with an array of solid-phase bound primers of known sequence and position. The reaction is conducted in the presence of labeled nucleotides or labeled dideoxy nucleotides (Chetverin, A. B. et al. (1994) “OLIGONUCLEOTIDE ARRAYS: NEW CONCEPTS AND POSSIBITIIES,” Bio/Technology 12:1093-1099; Macevicz (U.S. Pat. No. 5,002,867); Beattie, W. G. et al. (1995) “HYBRIDIZATION OF DNA TARGETS TO GLASS-TETHERED OLIGONUCLEOTIDE PROBES,” Molec. Biotech. 4:213-225; Boyce-Jacino et al. (U.S. Pat. No. 6,294,336); Head et al. (U.S. Pat. No. 6,322,968); Head et al. (U.S. Pat. No. 6,337,188). Caskey, C. et al. has described a method of analyzing a polynucleotide of interest using one or more sets of consecutive oligonucleotide primers differing within each set by one base at the growing end thereof (Caskey, C. et al. (WO 95/00669)). The oligonucleotide primers are extended with a chain terminating nucleotide and the identity of each terminating nucleotide is determined.

Pastinen, T. et al. has described a method for the multiplex detection of mutations wherein the mutations are detected by extending immobilized primers, that anneal to the template sequences immediately adjacent to the mutant nucleotide positions, with a single labeled dideoxynucleotide using a DNA polymerase (Pastinen, T. et al. (1997) “MINISEQUENCING: A SPECIFIC TOOL FOR DNA ANALYSIS AND DIAGNOSTICS ON OLIGONUCLEOTIDE ARRAYS,” Genome Res. 7:606-614). In this method, the oligonucleotide arrays were prepared by coupling one primer per mutation to be detected on a small glass area. Pastinen, T. et al. has also described a method to detect multiple single nucleotide polymorphisms in an undivided sample (Pastinen, T. et al. (1996) “MULTIPLEX, FLUORESCENT, SOLID-PHASE MINISEQUENCING FOR EFFICIENT SCREENING OF DNA SEQUENCE VARIATION,” Clin. Chem. 42:1319-1397). According to this method, the amplified DNA templates are first captured onto a manifold and then, with multiple minsequencing primers, single nucleotide extension reactions are carried out simultaneously with fluorescently labeled dideoxynucleotides.

Jalanko, A. et al. has described the application of solid-phase minisequencing methods to the detection of a mutation causing cystic fibrosis (Jalanko, A. et al. (1992) “SCREENING FOR DEFINED CYSTIC FIBROSIS MUTATIONS BY SOLID-PHASE MINISEQUENCING,” Clin. Chem. 38:39-43). In this method, an amplified DNA molecule that is biotinylated at its 5′ terminus is bound to a solid phase and denatured. A detection primer, which hybridizes immediately before the putative mutation, is hybridized to the immobilized single stranded template and elongated with a single, labeled deoxynucleoside residue. Shumaker, J. M. et al. has described another solid phase primer extension method for mutation detection (Shumaker, J. M. et al. “MUTATION DETECTION BY SOLID PHASE PRIMER EXTENSION,” (1996) Hum. Mutation 7:346-354). In this method, template DNA is annealed to an oligonucleotide array, extended with ³²P dNTPs and analyzed with a phosphoimager.

Sequencing determination methods have also been developed that rely on the extent of hybridization between a probe and a template molecule (Drmanac, R. et al. (2002) “SEQUENCING BY HYBRIDIZATION (SBH): ADVANTAGES, ACHIEVEMENTS, AND OPPORTUNITIES,” Adv. Biochem. Eng. Biotechnol. 77:75-101; Drmanac, R. et al. (2001) “SEQUENCING BY HYBRIDIZATION ARRAYS,” Methods Molec. Biol. 170:39-51; Gabig, M. et al. (2001) “AN INTRODUCTION TO DNA CHIPS: PRINCIPLES, TECHNOLOGY, APPLICATIONS AND ANALYSIS,” Acta Biochim. Pol. 48:615-22). Drmanac, R. T., for example, has described a method for sequencing nucleic acid by hybridization using nucleic acid segments on different sectors of a substrate and probes that discriminate between a one base mismatch (Drmanac, R. T. (EP 797683)). Gruber, L. S. has described a method for screening a sample for the presence of an unknown sequence using hybridization sequencing (Gruber, L. S. (EP 787183)). Landegren, U. et al. have described the “Oligonucleotide Ligation Assay” (“OLA”) (Landegren, U. et al. (1988) “LIGASE-MEDIATED GENE DETECTION TECHNIQUE,” Science 241:1077-1080) as being capable of detecting single nucleotide polymorphisms. The OLA protocol uses two oligonucleotides which are designed to be capable of hybridizing to abutting sequences of a single strand of a target. One of the oligonucleotides is biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of the polymerase chain reaction (PCR) and OLA (Nickerson, D. A. et al. (1990) “AUTOMATED DNA DIAGNOSTICS USING AN ELISA-BASED OLIGONUCLEOTIDE LIGATION ASSAY,” Proc. Natl. Acad. Sci. (U.S.A.) 87:8923-8927). In this method, PCR is used to achieve the exponential amplification of target DNA, which is then detected using OLA. In addition to requiring multiple, and separate processing steps, one problem associated with such combinations is that they inherit all of the problems associated with PCR and OLA.

Exonucleases are enzymes that degrade nucleic acid molecules from either their 3′ or 5′ terminus. As indicated above, exonucleases have been used to facilitate DNA sequencing (Mundy, C. R. (U.S. Pat. No. 4,656,127)). Jett et al. have proposed the use of exonucleases to accomplish the stepwise degradation of a target nucleic acid molecule and the sequential analysis of each, released nucleotide (Jett, J. H. et al. (1989) “HIGH-SPEED DNA SEQUENCING: AN APPROACH BASED UPON FLUORESCENCE DETECTION OF SINGLE MOLECULES,” J Biomolecular Structure & Dynamics 7:301-309; Jett et al. (WO 89/03432)). Koster (U.S. Pat. No. 6,140,053; U.S. Pat. No. 6,074,823) disclose a sequencing strategy that uses mass spectroscopy to analyze the differences in mass of the fragments obtained through exonuclease digestion. Murtagh (U.S. Pat. No. 5,688,669) describes the use of the 3′ to 5′ exonuclease, Exonuclease III, to digest a target DNA molecule in to fragments and then determine their sequence via hybridization to complementary probes.

Labeit, S. et al. have disclosed a sequencing method in which four separate primer extension reactions are conducted, each in the presence of a different phosphothioated deoxynucleoside and three conventional nucleotides (Labeit, S. et al. “LABORATORY METHODS, A NEW METHOD OF DNA SEQUENCING USING DEOXYNUCLEOSIDE A-TRIPHOSPHATES,” DNA 4:173-177). The primer extension reactions are then incubated in the presence of Exonuclease III. Since exonucleases cannot cleave phosphothioated nucleotides, treatment with the exonuclease results in the production of a nested set of fragments each containing a phosphothioated nucleoside at its 3′ terminus (Putney, S. D. et al. (1981) “A DNA FRAGMENT WITH AN ALPHA-PHOSPHOROTHIOATE NUCLEOTIDE AT ONE END IS ASYMMETRICALLY BLOCKED FROM DIGESTION BY EXONUCLEASE III AND CAN BE REPLICATED IN VIVO,” Proc Natl Acad Sci (USA) 78:7350-7354; Nakamaye, K. L. et al. (1988) “DIRECT SEQUENCING OF POLYMERASE CHAIN REACTION AMPLIFIED DNA FRAGMENTS THROUGH THE INCORPORATION OF DEOXYNUCLEOSIDE α-THIOTRIPOSPHATES,” Nucleic Acids Res. 16:9947-9959). The sequences of the molecules can be determined using gel electrophoresis methods.

Iyyalasomayazula (U.S. Pat. No. 6,165,726) describes the biotin labeling of molecules for sequencing, and the use of immobilized streptavidin to capture such molecules.

Despite the development of all such methods, a need continues to exist for an improved, rapid, and sensitive method for sequencing DNA that avoids the need for specialized enzymes and procedures. The present invention is directed to this and other goals.

### SUMMARY OF THE INVENTION

Current DNA sequencing strategies revolve around the use of primer extension on a target or template DNA. In the most widely employed method, all four deoxynucleotides are included in the polymerization reaction as well as 4 dideoxynucleotides. Since automated DNA sequencers have the ability to distinguish different dyes, a complete sequencing reaction can be performed in a single tube if each dideoxynucleotide is labeled with a different dye. The resulting DNA fragments are the products of primer extensions from a single primer, but termination results from the incorporation of 4 different dye labeled dideoxynucleotides. The present invention is intended to provide an alternative sequencing method, and, in preferred embodiments produces 4 different dye labeled DNA fragments by a novel approach that employs more robust chemistries and involves less stringent requirements for “special” polymerases

In detail, the invention provides a method for determining the sequence of a region of one strand of a double-stranded nucleic acid target molecule, wherein the method comprises incubating the nucleic acid target molecule in the presence of an exonuclease activity, a polymerase activity and four differentially detectable, exonuclease activity-resistant, chain terminator nucleotide species.

The invention particularly concerns the embodiment of such methods wherein four differentially detectable, exonuclease activity-resistant, chain terminator nucleotide species are employed. The invention further concerns the embodiment of such methods wherein at least one of the four differentially detectable, exonuclease activity-resistant, chain terminator nucleotide species is fluorescently labeled. The invention further concerns the embodiment of such methods wherein the four differentially detectable, exonuclease activity-resistant, chain terminator nucleotide species are fluorescently labeled.

The invention further concerns the embodiment of such methods wherein the double-stranded nucleic acid target molecule possesses only one 3′ terminus that is a substrate for the exonuclease activity. The invention further concerns the embodiment of such methods wherein the double-stranded nucleic acid target molecule possesses a 3′ terminus that extends beyond the 5′ terminus of the opposite strand. The invention further concerns the embodiment of such methods wherein the double-stranded nucleic acid target molecule possesses a 3′ terminus that is sterically blocked from exonuclease activity degradation. The invention additionally concerns the embodiment of such methods wherein both strands of the double-stranded nucleic acid target molecule possess a 3′ terminus that is a substrate for the exonuclease activity.

The invention further concerns the embodiments of such methods wherein one 5′ terminus or both 5′ termini of the double-stranded nucleic acid target molecule possesses a haptenic group, especially wherein the haptenic group is biotin.

The invention further concerns a method for determining the nucleotide sequence of a region of a double-stranded nucleic acid target molecule, wherein the method comprises the steps:

(A) incubating a preparation of the double-stranded target molecule in the presence of a 3′ to 5′ exonuclease activity, wherein the double-stranded nucleic acid target molecule possess at least one 3′ terminus that is a substrate for the exonuclease activity, wherein the incubation is conducted under conditions sufficient to permit the exonuclease activity to produce a nested population of double-stranded nucleic acid target molecule having at least one degraded 3′ termini;

(B) incubating the nested population of double-stranded nucleic acid target molecule in the presence of a polymerase activity and at least one detectably labeled, exonuclease activity-resistant, chain terminator nucleotide species, wherein the incubation is conducted under conditions sufficient to permit the polymerase activity to mediate the template-dependent incorporation of one of the nucleotide species onto the 3′ terminus of a nucleic acid target molecule whose 3′ terminus was degraded by the exonuclease activity; and

(C) determining the identity of the differentially detectable, exonuclease activity-resistant, chain terminator nucleotide species incorporated onto the 3′ terminus at the selected region.

The invention further concerns the embodiment of such method wherein the steps A and B are conducted simultaneously, and wherein the conditions employed are sufficient to permit the exonuclease activity to degrade the substrate termini and sufficient to permit the polymerase activity to mediate the template-dependent incorporation of the nucleotide species.

The invention particularly concerns the embodiment of such methods wherein four differentially detectable, exonuclease activity-resistant, chain terminator nucleotide species are employed. The invention further concerns the embodiment of such methods wherein at least one of the four differentially detectable, exonuclease activity-resistant, chain terminator nucleotide species is fluorescently labeled. The invention further concerns the embodiment of such methods wherein the four differentially detectable, exonuclease activity-resistant, chain terminator nucleotide species are fluorescently labeled.

The invention further concerns the embodiment of such methods wherein the double-stranded nucleic acid target molecule possesses only one 3′ terminus that is a substrate for the exonuclease activity. The invention further concerns the embodiment of such methods wherein the double-stranded nucleic acid target molecule possesses a 3′ terminus that extends beyond the 5′ terminus of the opposite strand. The invention further concerns the embodiment of such methods wherein the double-stranded nucleic acid target molecule possesses a 3′ terminus that is sterically blocked from exonuclease activity degradation. The invention additionally concerns the embodiment of such methods wherein both strands of the double-stranded nucleic acid target molecule possess a 3′ terminus that is a substrate for the exonuclease activity.

The invention further concerns the embodiments of such methods wherein one 5′ terminus or both 5′ termini of the double-stranded nucleic acid target molecule possesses a haptenic group, especially wherein the haptenic group is biotin.

The invention also concerns an in vitro composition comprising a double-stranded nucleic acid target molecule, an exonuclease activity, a polymerase activity and four differentially detectable, exonuclease activity-resistant, chain terminator nucleotide species.

The invention further concerns the embodiments of such composition wherein at least one of the four differentially detectable, exonuclease activity-resistant, chain terminator nucleotide species is fluorescently labeled. The invention further concerns the embodiments of such compositions wherein the four differentially detectable, exonuclease activity-resistant, chain terminator nucleotide species are fluorescently labeled. The invention further concerns the embodiments of such compositions wherein at least one 5′ terminus or wherein both 5′ termini of the double-stranded nucleic acid target molecule possesses a haptenic group, especially wherein the haptenic group is biotin.

The invention further concerns a composition comprising four differentially detectable, exonuclease activity-resistant, chain terminator nucleotide species. The invention further concerns the embodiments of such composition wherein at least one of the four differentially detectable, exonuclease activity-resistant, chain terminator nucleotide species is fluorescently labeled. The invention further concerns the embodiments of such compositions wherein the four differentially detectable, exonuclease activity-resistant, chain terminator nucleotide species are fluorescently labeled.

The invention further concerns a kit specially adapted to facilitate the sequencing of a target nucleic acid molecule, the kit comprising a first container comprising a primer A, a second container comprising a primer B, and a third container containing an exonuclease activity, wherein the primers A and B mediate the amplification of a double-stranded nucleic acid molecule comprising the target nucleic acid molecule, and wherein at least one of the primer A or the primer B possesses a 5′ terminus having at least one modified nucleotide.

The invention further concerns the embodiments of such kit wherein the modified nucleotide is a ribonucleotide, a dUridine nucleotide, a phosphothioate nucleotide, or a biotin-derivatized nucleotide. The invention further concerns the embodiments of such kits wherein the kit further comprises a fourth container containing four detectably labeled, exonuclease activity-resistant, chain terminator nucleotide species, and especially wherein the four detectably labeled, exonuclease activity-resistant, chain terminator nucleotide species are fluorescently labeled.

The invention further concerns a sequenator, comprising an apparatus for determining the identity of fluoresecently labeled exonuclease activity-resistant, chain terminator nucleotide species incorporated onto the 3′ termini of a nucleic acid target molecule whose 3′ terminus was degraded by the exonuclease; and then extended by a template-dependent polymerase to incorporate the fluorescently labeled nucleotide species.

### BRIEF DESCRIPTION OF THE FIGURES

FIG. 1 illustrates the use of a preferred embodiment of the invention to sequence double-stranded DNA. In the figure, B represents Biotin; closed solid circles, striped circles, open circles, and dot-filled circles represent four differentially detectable exonuclease activity-resistant, chain terminator nucleotide species.

FIG. 2 illustrates the use of the present invention to sequence one or both strands of a double-stranded nucleic acid target molecule. In the figure closed solid circles, striped circles, open circles, and dot-filled circles represent four differentially detectable exonuclease activity-resistant, chain terminator nucleotide species.

### DESCRIPTION OF THE PREFERRED EMBODIMENTS

The invention relates to methods, compositions, kits and apparati for sequencing nucleic acid molecules, including RNA or DNA. The invention particularly concerns the incubation of reagents in the presence of exonuclease activity, especially in concert with a polymerase activity, in order to mediate such sequencing.

The term “exonuclease activity,” as used herein refers to an enzymatic activity (or a chemical process equivalent thereof) that is capable of removing a nucleotide from the terminus of a nucleic acid molecule. Preferred exonuclease activities can remove nucleotides from the 3′ termini of a nucleic acid molecule. Examples of such preferred 3′ to 5′ exonuclease activities include the 3′ to 5′ exonuclease activity of snake venom phosphodiesterase, the 3′ to 5′ exonuclease activity of spleen phosphodiesterase, the 3′ to 5′ exonuclease activity of Bal-31 nuclease, the 3′ to 5′ exonuclease activity of E. coli exonuclease I, the 3′ to 5′ exonuclease activity of E. coli exonuclease VII, the 3′ to 5′ exonuclease activity of Mung Bean Nuclease, the 3′ to 5′ exonuclease activity of S1 Nuclease, the 3′ to 5′ exonuclease activity of E. coli DNA polymerase I, the 3′ to 5′ exonuclease activity of the Klenow fragment of DNA polymerase I, the 3′ to 5′ exonuclease activity of T4 DNA polymerase, the 3′ to 5′ exonuclease activity of T7 DNA polymerase, the 3′ to 5′ exonuclease activity of E. coli exonuclease III, the 3′ to 5′ exonuclease activity of k exonuclease, the 3′ to 5′ exonuclease activity of Pyrococcus species GB-D DNA polymerase and the 3′ to 5′ exonuclease activity of Thermococcus litoralis DNA polymerase. E. coli exonuclease III is particularly preferred for use in the present invention.

As used herein, the term “polymerase activity” refers to an enzymatic activity (or a chemical process equivalent thereof) that is capable of extending the terminus of a nucleic acid molecule in a template-dependent manner (e.g., by mediating the incorporation of a nucleotide onto the 3′ terminus of a primer molecule hybridized to a complementary template). Polymerase activities relevant to the present invention include the polymerase activity of thermostable polymerases (such as Accuzyme, Biolase Diamond polymerase (Bioline); Tbr Polymerase, Tfl polymerase, Tsp B polymerase (BioNexus; www.bionexus.net); Thermus polymerase (Chimerx; www.chimerx.com); MasterAmp Amplitherm polymerase, MasterAmp Tfl polymerase (Epicentre; www.epicentre.com); DyN/Azyme I and II polymerase (Finnzymes; www.finnzymes.com); Accutherm polymerase (GeneCraft; www.genecraft.de); Taq polymerase, ThermalAce polymerase (Invitrogen; www.invitrogen.com); VentR (exo-) polymerase, VentR polymerase, Deep VentR (exo-) polymerase, Deep VentR polymerase, Bst polymerase (New England Biolabs; www.neb.com); Pfu Polymerase, Tfl Polymerase, Tli Polymerase (Promega; www.promega.com); Pyra exo(-) polymerase, Tfu Polymerase (Qbiogene; www.qbiogene.com); Tgo Polymerase, Pwo Polymerase (Roche Molecular Biochemicals; biochem.roche.com); Pfu native polymerase, Pfu recombinant polymerase, PfuTurbo polymerase (Stratagene; www.stratagene.com); Pwo polymerase (ThermoHybaid; www.thermohybaid.com), etc., as well as the polymerase activity of non-thermostable polymerases (such as DNA polymerase III from E. coli, Klenow polymerase, T4 polymerase, T7 polymerase, Φ29 polymerase, etc.).

In accordance with the principles of the present invention, suitable polymerase activities are possessed by polymerases that are able to mediate the incorporation into nucleic acid molecules of nucleotides and nucleotide analogs that are not substrates of exonuclease activity. Preferably, such polymerase activities will be capable of mediating the incorporation of modified nucleotides (e.g., methylated nucleotides, phosphothioated nucleotides, ribonucleotides), and especially chain terminator nucleotide species (such as dideoxynucleotides), and/or labeled nucleotides (such as those possessing fluorescent (e.g., α-thio dye terminators, borate dye terminators, etc.), radioactive, paramagnetic, chemiluminescent, enzymatic, haptenic, antigenic, etc., labels.

In preferred embodiments, the invention is directed to a method for sequencing nucleic acid molecules in which the individual molecules of a preparation of target molecules is subjected to 3′ exonuclease activity-mediated digestion, and to polymerase activity-mediated extension in the presence of exonuclease activity-resistant chain-terminating nucleotides or nucleotide derivatives. The invention contemplates that the exonuclease activity treatment may precede, or may be accomplished simultaneously with, the polymerase activity-mediated extension reaction.

As indicated above, the preferred embodiments of the present invention employs differentially detectable, exonuclease activity-resistant, chain-terminating nucleotides or nucleotide derivatives. Any modification that renders the incorporated nucleotide “chain terminating” may be employed. Particularly preferred are the dideoxynucleotides whose ribosyl moiety lacks a 3′ hydroxyl group.

Depending upon the desired application, one, two, three or four different exonuclease activity-resistant chain-terminating nucleotides or nucleotide derivatives may be employed. For example, determinations of single nucleotide polymorphisms may be accomplished using one, two, three or four different exonuclease activity-resistant chain-terminating nucleotides or nucleotide derivatives. Applications involving the sequencing of DNA, will preferably entail the use of four different exonuclease activity-resistant chain-terminating nucleotides or nucleotide derivatives will be employed.

Preferably, the employed exonuclease activity-resistant chain-terminating nucleotides will be differentially detectable. As used herein, the term “differentially detectable” denotes the use or presence of a label that that can be detected even in the presence of another label. Such differentially detectability can be attained in a variety of ways. For example, different classes of labels (e.g., some radioactive, some fluorescent, etc.) may be used. More preferably, the differentially detectable labels will be of the same class (e.g., all radioactive, all fluorescent, etc.). Fluorescent labels are particularly preferred. For example, nucleotides can be labeled with FAM (emission at 518 nm), HEX (emission at 556 nm), Alexa 594 (emission at 612 nm) and Cy5 (emission at 670 nm) to provide four differentially detectable nucleotides.

A large number of fluorescent nucleotide analogues are suitable for use in the methods and compositions of this invention (see, e.g., Kricka, L. J. (2002) “STAINS, LABLELS AND DETECTION STRATEGIES FOR NUCLEIC ACIDS ASSAYS,” Ann. Clin. Biochem. 39:114-129). Suitable fluorescent labels include FAM (e.g., 6-FAM, etc.), HEX, Cy5, Cy5.5, Cy3, JOE, TAMRA (e.g., 6-TAMRA, 5-TAMRA, etc.), MANT, BODIPY (e.g., BODIPY FL-14, BODIPY TR-14, BODIPY TMR-14, BODIPY R6G, etc.), Alexa (e.g., Alexa 430, Alexa 488, Alexa 546, Alexa 594, etc.), Texas Red (e.g., Texas Red-5, etc.), Cascade Blue, Fluorescein (e.g., Fluorescein-12, etc.), TET (e.g., Tetramethylrhodamine-6, etc.), rhodamine (e.g., rhodamine red, rhodamine green, rhodamine 6G and ROX (e.g., 6-ROX, etc.). Rhodamine 110; rhodol; cyanine; coumarin or a fluorescein compound (rhodamine 110, rhodol, or fluorescein compounds that have a 4′ or 5′ protected carbon) may be employed. Preferred examples of such compounds include 4′(5′)thiofluorescein, 4′(5′)-aminofluorescein, 4′(5′)-carboxyfluorescein, 4′(5′)-chlorofluorescein, 4′(5′)-methylfluorescein, 4′(5′)-sulfofluorescein, 4′(5′)-aminorhodol, 4′(5′)-carboxyrhodol, 4′(5′)-chlororhodol, 4′(5′)-methylrhodol, 4′(5′)-sulforhodol; 4′(5′)-aminorhodamine 110, 4′(5′)-carboxyrhodamine 110, 4′(5′)-chlororhodamine 110, 4′(5′)-methylrhodamine 110, 4′(5′)-sulforhodamine 110 and 4′(5′)thiorhodamine 110. “4′(5′)” means that at the 4 or 5′ position the hydrogen atom on the carbon atom is substituted with a specific organic group or groups as previously listed. A 7-Amino, or sulfonated coumarin derivative may likewise be employed. Fluorescein-12-dUTP, Rhodamine-5-dUTP, and Coumarin-6-dUTP may be employed.

Any modification sufficient to render the incorporated nucleotide resistant to exonuclease activity treatment may be employed. Preferred exonuclease activity-resistant derivatives will possess α-thio or α-P-borano groups.

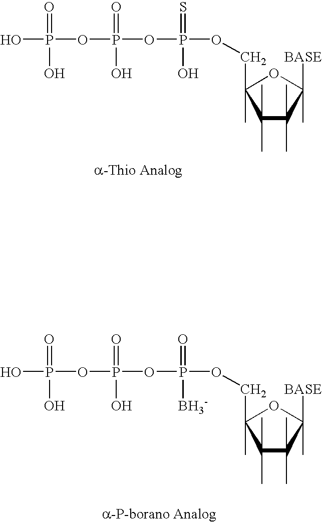

The exonuclease activity treatment degrades the target molecules from their 3′ termini, and results in the creation of a set of target molecule fragments having nested 3′ termini. The polymerase activity treatment results in the installation of an exonuclease activity-resistant chain-terminating nucleotide at this terminus. Thus, the net consequence of the exonuclease activity/polymerase activity reactions is the creation of a nested set of target molecule fragments having a labeled exonuclease activity-resistant chain-terminating nucleotide or nucleotide analog at their 3′ termini.

Once such a nested set of target molecule fragments has been produced, its members can be retrieved and analyzed to determine the identity of the 3′ terminal nucleotides. For example, the molecules can be subjected to gel electrophoresis. The label (of the incorporated exonuclease activity-resistant chain-terminating nucleotide) associated with a particular band in the gel identifies the 3′ terminal nucleotide present in the molecules that make up that band. By comparing multiple bands, the sequence of the original target molecule can be readily deduced. Although such analysis may be done manually, it is preferable to employ an automated sequencer for this purpose. The CEQ200XL and CEQ8000 Genetic Analysis Systems (Beckman-Coulter, Inc.) are particularly preferred, especially in concert with the Biomek® 2000 Laboratory Automation Workstation (Beckman-Coulter, Inc.). Although the use of electrophoresis is a preferred method for determining the sequence of the labeled molecules, other methods, such as mass spectroscopy, laser desorption mass spectrometry (LDMS), MALDI-TOF MS, hybridization to ordered arrays, flow cytometry, micro-chi[separation, etc. (Dovichi, N. J. et al. (2001) “DNA SEQUENCING BY CAPILLARY ARRAY ELECTROPHORESIS,” Methods Molec. Biol. 167:225-39; Huber, C. G. et al. (2001) “ANALYSIS OF NUCLEIC ACIDS BY ON-LINE LIQUID CHROMATOGRAPHY-MASS SPECTROMETRY,” Mass Spectrom. Rev. 20:310-343; Buchholz, B. A. et al. (2001) “THE USE OF LIGHT SCATTERING FOR PRECISE CHARACTERIZATION OF POLYMERS FOR DNA SEQUENCING BY CAPILLARY ELECTROPHORESIS,” Electrophoresis 22:4118-28; Mitnik, L. et al. (2001) “RECENT ADVANCES IN DNA SEQUENCING BY CAPILLARY AND MICRODEVICE ELECTROPHORESIS,” Electrophoresis 22:4104-17; Bonk, T. et al. (2001) “MALDI-TOF-MS ANALYSIS OF PROTEIN AND DNA,” Neuroscientist 7:6-12; Gawron, A. J. et al. (2001) “MICROCHIP ELECTROPHORETIC SEPARATION SYSTEMS FOR BIOMEDICAL AND PHARMACEUTICAL ANALYSIS,” Eur. J. Pharm. Sci. 14(1):1-12).

The preferred embodiments of the present invention thus enable multiple sequencing reactions (i.e., reactions involving the incorporation of different nucleotide species to be performed simultaneously in a single reaction vessel. In its preferred embodiments, the invention differs from conventional dideoxynucleotide sequencing in that it can be conducted in the absence or substantial absence of non-chain termination nucleotide triphosphates. In preferred embodiments of the invention, thermostabile polymerase activities are not required and the use of modified polymerases can be minimized or avoided. Additionally, thermocycling is not required (thereby obviating “heated lid” or evaporation issues that affect conventional dideoxynucleotide sequencing, while providing more rapid sequencing with higher throughput). Additionally, in preferred embodiments of the present invention, the denaturation of template, in order for primer to gain access to the template, is unnecessary.

In a preferred embodiment, the methods of the present invention permit the sequencing of both strands of a double-stranded nucleic target molecules. In a further preferred embodiment, one strand of the produced nested set of labeled oligonucleotides will additionally be specially modified so as to facilitate their recovery and analysis. In yet another preferred embodiment, such modification is accomplished by modifying the target molecule to contain a haptenic group. Such a modification permits the oligonucleotides to be preferentially recovered and/or immobilized by “agents” that bind to the haptenic group. Such modification may be introduced at any region of the target molecule, but will preferably be provided at a site at or near the target molecule's 5′ terminus. Suitable haptenic groups may be biotin groups, antigens, binding ligands, etc., where the “agent” is avidin (or streptavidin, etc.), or an antibody, receptor, or binding partner that preferentially binds to the employed haptenic group. In a further preferred embodiment, such modification is achieved by forming the target molecule from the template-mediated extension of a primer molecule whose 5′ terminus has been modified with the haptenic group.

In a particularly preferred embodiment, a preparation of a single-stranded target nucleic acid molecule is prepared having a biotin moiety at its 5′ terminus. The preparation is incubated in the presence of an exonuclease activity (e.g., E. coli Exonuclease III) and a polymerase activity (e.g., Klenow polymerase), and four differentially detectable, exonuclease activity-resistant, chain-terminating nucleotides under conditions sufficient to permit the exonuclease activity and polymerase activity reactions to proceed.

Reagents (such as EDTA, base, etc.) are added in amounts sufficient to terminate the reaction. The nucleic acid molecules are captured onto a streptavidin plate by incubating them in contact with the plate under suitable conditions (e.g., 25° C. for 0.5 h with occasional mixing). The plate is then washed with alkali (e.g., 0.1 M NaOH at 25° C. for 5 min), and is treated with formamide and heat (98% formamide containing 10 mM EDTA at 94° C. for 5 min.). The material is then loaded onto a gel, and is subjected to gel electrophoresis. The resulting bands are then analyzed to determine the identity of the labeled 3′ terminator nucleotide in each band, thereby providing the nucleotide sequence of the target molecule.

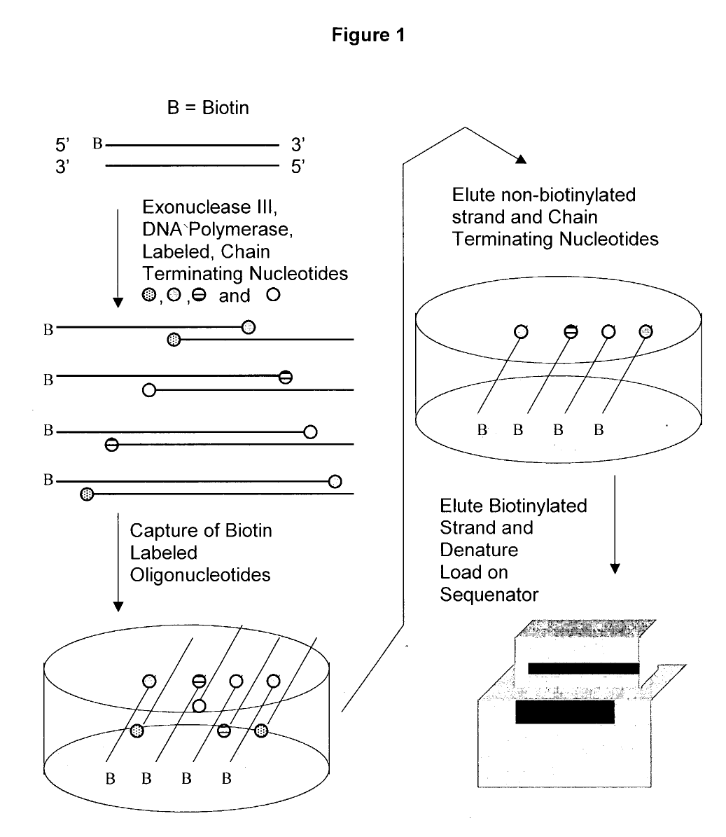

In a preferred example of such embodiment, illustrated in FIG. 1, one strand of a double-stranded nucleic target molecules will possess a biotin moiety (preferably at a site at or near the target molecule's 5′ terminus). The molecules can then be incubated in the presence of avidin (or more preferably streptavidin) that is preferably bound to a solid support. The target molecules can be recovered from such a support by treatment (such as heat denaturation) and then analyzed, as by gel electrophoresis to determine the identity of the incorporated labeled nucleotide.

The invention further contemplates additional preferred embodiments of such a method in which sequencing of only one strand can be accomplished. For example, exonuclease activity degradation of the 3′ terminus of the strand hybridized to the biotin-labeled strand can be sterically inhibited by incubating the double-stranded molecule in the presence of avidin or streptavidin. The binding of avidin or streptavidin to the biotin group inhibits the degradation of the 3′ terminus of the opposite strand, and thereby enables exonuclease activity to be conducted only or preferentially on one strand. Equivalently, a hapten or antigen may be used in place of biotin, and an antibody specific for such hapten or antigen may be employed in lieu of the avidin or streptavidin to sterically block the 3′ terminus of the opposite strand from exonuclease-mediated degradation.

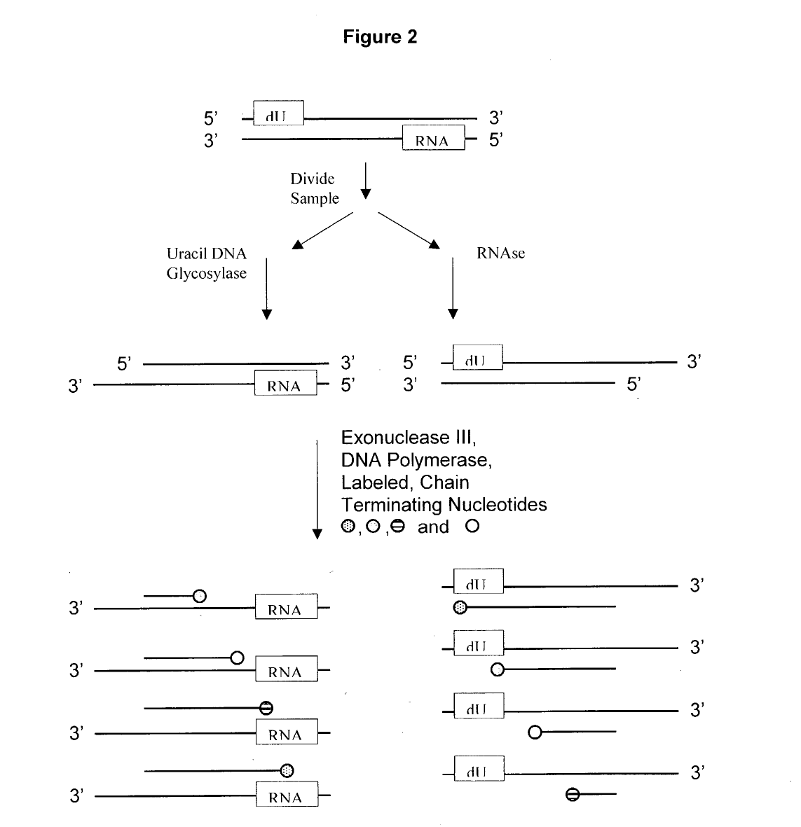

FIG. 2 illustrates one approach to such a preferred embodiment of the invention. Two primers (“primer A” and “primer B”) are employed to produce a preparation of target molecule. Primer A is designed to contain dUridine residue(s); primer B is designed to contain an oligoribonucleotide region. The preparation is divided and one aliquot treated with uracil DNA glycosylase; another aliquot is treated with RNAse or alkali. Uracil DNA glycosylase removes the dUridine base, but does not cleave the DNA backbone. Exonuclease activity (such as for example the exonuclease activity of Exonuclease III) cleaves the abasic site and thereby degrades the 5′ terminus of the primer A strand, thus exposing the 3′ terminus of the primer B strand. The RNAse or alkali treatment degrades the 5′ terminus of the primer B strand, thus exposing the 3′ terminus of the primer A strand. Since exonuclease III does not degrade an exposed 3′ terminus, such action causes the primer B strand of the Uracil DNA glycosylase-treated preparation and the primer A strand of the RNAse or alkali-treated preparation to be resistant to exonuclease action. Incubation in the presence of an exonuclease activity, a polymerase activity and differentially detectable, exonuclease activity-resistant, chain-terminating nucleotides or nucleotide derivatives thus permits the methods of the present invention to sequence the primer A strand of the uracil DNA glycosylase-treated preparation and the primer B strand of the RNAse or alkali-treated preparation. Other enzymatic activities may optionally be added to facilitate any or all of the above reactions.

In an alternative approach, the target molecule is formed through the extension of two primers in a reaction that includes the provision of phophothioate nucleotides, which are resistant to exonuclease activity. Such a reaction leads to the incorporation of phophothioate nucleotides into both primers. One primer (“primer A”) would preferably contain 4 phosphothioates toward its 3′ end. The other primer (“primer B”) would contain phosphothioates on its 5′ end. Addition of a 5′ to 3′ exonuclease would degrade the all “normal” phophodiester 5′ ends (alternatively the molecules could be formed with phosphorthioates, and treated with 2-iodoethanol and/or 2,3-epoxy-1-propanol to cleave the phosphorthioate nucleotides). The 5′ terminus of the strand primed from primer A would be degraded to expose the 3′ terminus of the other strand; the 5′ terminus of the strand primed from primer B would not be degraded. Since a single-stranded 3′ terminus is not susceptible to Exonuclease III activity, treatment with exonuclease would degrade the 5′ terminus of the primer A strand, and thereby render the 3′ terminus of the primer B strand resistant to exonuclease degradation. Thus, only the primer A strand would be sequenced in the reactions of the present invention.

Equivalently, ribonucleotides (or a primer containing an oligo-ribonucleotide region) can be employed in lieu of phosphothioate nucleotides. In a preferred embodiment of such an approach, the target molecules are subjected to treatment with RNAse or alkali so as to degrade the ribonucleotide portions of the target. By employing a “primer A” containing ribonucleotides toward its 3′ end and a “primer B” containing ribonucleotides on its 5′ end, treatment with RNAse or alkali would degrade the 5′ terminus of the primer A strand, and thereby render the 3′ terminus of the primer B strand resistant to exonuclease degradation. Only the primer A strand would be sequenced in the reactions of the present invention.

In a further embodiment, the target molecules can be formed from the extension of a pair of primers, one of which has a restriction site not contained elsewhere in the sequence of the target that, when cleaved generates a 3′ overhang. Treatment with the restriction endonuclease that recognizes such site thus renders the strand possessing the overhang resistant to sequencing in accordance with the methods of the present invention. In a further embodiment, the target molecules can be formed from the extension of a pair of primers, each having a unique restriction site not contained elsewhere in the sequence of the target molecule that, when cleaved generates a 3′ overhang. This embodiments permits the two strands of the target molecule to be separately sequenced, by treatment with one restriction endonuclease, sequencing of the exonuclease sensitive strand, treatment with the second restriction endonuclease, and sequencing of the second strand.

The methods of the present invention may be used to sequence any nucleic acid molecules, including nucleic acid molecules of mammalian origin (especially human, simian, canine, bovine, ovine, feline, and rodent), of plant origin, or of bacterial or lower eukaryotic origin. The methods of the present invention may be used to sequence nucleic acid molecules of pathogens (including bacterial, yeast, fungal and viral pathogens).

The present invention also concerns compositions and kits specially adapted to facilitate the above described methods. Exemplary compositions include preparations of nucleotides that lack conventional (non-chain terminating) nucleotides but contain four differentially detectable exonuclease resistant, chain terminator nucleotide species, primers containing modified nucleotides or regions that can be employed to produce desired target molecules, and reagents and enzymes adapted to act upon such primers to permit the sequencing of one strand of a nucleic acid molecule.

The present invention also concerns apparati, such as automated sequenators that have been specially adapted to conduct the methods of the present invention.

All publications and patent applications mentioned in this specification are herein incorporated by reference to the same extent as if each individual publication or patent application had been specifically and individually indicated to be incorporated by reference. The discussion of the background to the invention herein is included to explain the context of the invention. Such explanation is not an admission that any of the material referred to was published, known, or part of the prior art or common general knowledge anywhere in the world as of the priority date of any of the aspects listed above.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method for determining the sequence of a region of one strand of a double-stranded nucleic acid target molecule, wherein said method comprises incubating said nucleic acid target molecule in the presence of an exonuclease activity, a polymerase activity and four differentially detectable, exonuclease activity-resistant, chain terminator nucleotide species.

2. The method of claim 1, wherein four differentially detectable, exonuclease activity-resistant, chain terminator nucleotide species are employed.

3. The method of claim 2, wherein at least one of said four differentially detectable, exonuclease activity-resistant, chain terminator nucleotide species is fluorescently labeled.

4. The method of claim 3, wherein said four differentially detectable, exonuclease activity-resistant, chain terminator nucleotide species are fluorescently labeled.

5. The method of claim 1, wherein said double-stranded nucleic acid target molecule possesses only one 3′ terminus that is a substrate for said exonuclease activity.

6. The method of claim 5, wherein said double-stranded nucleic acid target molecule possesses a 3′ terminus that extends beyond the 5′ terminus of the opposite strand.

7 The method of claim 5, wherein said double-stranded nucleic acid target molecule possesses a 3′ terminus that is sterically blocked from exonuclease activity degradation.

8. The method of claim 1, wherein both strands of said double-stranded nucleic acid target molecule possess a 3′ terminus that is a substrate for said exonuclease activity.

9. The method of claim 1, wherein one 5′ terminus of said double-stranded nucleic acid target molecule possesses a haptenic group.

10. The method of claim 9, wherein said haptenic group is biotin.

11. The method of claim 1, wherein both 5′ termini of said double-stranded nucleic acid target molecule possess a haptenic group.

12. The method of claim 11, wherein said haptenic group is biotin.

13. A method for determining the nucleotide sequence of a region of a double-stranded nucleic acid target molecule, wherein said method comprises the steps:
(A) incubating a preparation of said double-stranded target molecule in the presence of a 3′ to 5′ exonuclease activity, wherein said double-stranded nucleic acid target molecule possess at least one 3′ terminus that is a substrate for said exonuclease activity, wherein said incubation is conducted under conditions sufficient to permit said exonuclease activity to produce a nested population of double-stranded nucleic acid target molecule having at least one degraded 3′ termini;
(B) incubating said nested population of double-stranded nucleic acid target molecule in the presence of a polymerase activity and at least one detectably labeled, exonuclease activity-resistant, chain terminator nucleotide species, wherein said incubation is conducted under conditions sufficient to permit said polymerase activity to mediate the template-dependent incorporation of one of said nucleotide species onto the 3′ terminus of a nucleic acid target molecule whose 3′ terminus was degraded by said exonuclease activity; and
(C) determining the identity of the differentially detectable, exonuclease activity-resistant, chain terminator nucleotide species incorporated onto said 3′ terminus at said selected region.

14. The method of claim 13, wherein said steps A and B are conducted simultaneously, and wherein said conditions employed are sufficient to permit said exonuclease activity to degrade said substrate termini and sufficient to permit said polymerase activity to mediate said template-dependent incorporation of said nucleotide species.

15. The method of claim 13, wherein four differentially detectable, exonuclease activity-resistant, chain terminator nucleotide species are employed.

16. The method of claim 13, wherein at least one of said four differentially detectable, exonuclease activity-resistant, chain terminator nucleotide species is fluorescently labeled.

17. The method of claim 16, wherein said four differentially detectable, exonuclease activity-resistant, chain terminator nucleotide species are fluorescently labeled.

18. The method of claim 13, wherein said double-stranded nucleic acid target molecule possesses only one 3′ terminus that is a substrate for said exonuclease activity.

19. The method of claim 18, wherein said double-stranded nucleic acid target molecule possesses a 3′ terminus that extends beyond the 5′ terminus of the opposite strand.

20. The method of claim 18, wherein said double-stranded nucleic acid target molecule possesses a 3′ terminus that is sterically blocked from exonuclease activity degradation.

21. The method of claim 13, wherein both strands of said double-stranded nucleic acid target molecule possess a 3′ terminus that is a substrate for said exonuclease activity.

22. The method of claim 13, wherein one 5′ terminus of said double-stranded nucleic acid target molecule possesses a haptenic group.

23. The method of claim 22, wherein said haptenic group is biotin.

24. The method of claim 13, wherein both 5′ termini of said double-stranded nucleic acid target molecule possess a haptenic group.

25. The method of claim 24, wherein said haptenic group is biotin.

26. An in vitro composition comprising a double-stranded nucleic acid target molecule, an exonuclease activity, a polymerase activity and four differentially detectable, exonuclease activity-resistant, chain terminator nucleotide species.

27. The in vitro composition of claim 13, wherein at least one of said four differentially detectable, exonuclease activity-resistant, chain terminator nucleotide species is fluorescently labeled.

28. The in vitro composition of claim 27, wherein said four differentially detectable, exonuclease activity-resistant, chain terminator nucleotide species are fluorescently labeled.

29. The in vitro composition of claim 13, wherein at least one 5′ terminus of said double-stranded nucleic acid target molecule possesses a haptenic group.

30. The in vitro composition of claim 29 wherein said haptenic group is biotin.

31. The in vitro composition of claim 13, wherein both 5′ termini of said double-stranded nucleic acid target molecule possesses a haptenic group.

32. The in vitro composition of claim 31, wherein said haptenic group is biotin.

33. A kit specially adapted to facilitate the sequencing of a target nucleic acid molecule, said kit comprising a first container comprising a primer A, a second container comprising a primer B, and a third container containing an exonuclease activity, wherein said primers A and B mediate the amplification of a double-stranded nucleic acid molecule comprising said target nucleic acid molecule, and wherein at least one of said primer A or said primer B possesses a 5′ terminus having at least one modified nucleotide.

34. The kit of claim 33, wherein said modified nucleotide is a ribonucleotide, a dUridine nucleotide, a phosphothioate nucleotide, or a biotin-derivatized nucleotide.

35. The kit of claim 33, wherein said kit further comprises a fourth container containing four detectably labeled, exonuclease activity-resistant, chain terminator nucleotide species.

36. The kit of claim 35, wherein said four detectably labeled, exonuclease activity-resistant, chain terminator nucleotide species are fluorescently labeled.

37. A sequenator, comprising an apparatus for determining the identity of fluoresecently labeled exonuclease activity-resistant, chain terminator nucleotide species incorporated onto the 3′ termini of a nucleic acid target molecule whose 3′ terminus was degraded by said exonuclease; and then extended by a template-dependent polymerase to incorporate said fluorescently labeled nucleotide species.
